(19) United States
(12) Patent Application Publication
(10) Pub. No.: US 2007/0179538 A1
(43) Pub. Date: Aug. 2, 2007

(54) Implantable subcutaneous medical device providing post-extra-systolic potentiation therapy

(76) Inventors: D. Curtis Deno, Andover, MN (US); William J. Havel, Maple Grove, MN (US)

Correspondence Address: MEDTRONIC, INC., 710 MEDTRONIC PARKWAY NE, MINNEAPOLIS, MN, 55432-9924, US

(21) Appl. No.: 11/342,785

(22) Filed: Jan. 30, 2006

(51) Int. Cl. A61N 1/00

(52) U.S. Cl. 607/5

(57) ABSTRACT

A subcutaneous implantable cardioverter-defibrillator provides post-shock post-extra-systolic potentiation therapy.

### BACKGROUND

The present invention pertains to medical devices and more particularly to implantable medical devices.

In some cases of mechanical cardiac dysfunction, PESP therapy has been found to restore an adequate response of the cardiac muscle to electrical depolarization, thereby increasing cardiac output (CO). PESP is achieved by delivering electrical pulses to a ventricle soon after a refractory period of a previous ventricular depolarization expires. As has been described in commonly assigned U.S. Pat. No. 5,213,098 and pre-grant publication U.S. 2004/0049235, which are hereby incorporated by reference in relevant part, PESP can increase the contractility of cardiac muscle for more vigorous pumping action in response to subsequent ventricular depolarization pulses.

Recent studies have shown that electrical defibrillation restoring normal sinus rhythm (NSR) after a prolonged period of fast ventricular tachyarrhythmia (VT) or ventricular fibrillation (VF) may not likewise restore adequate mechanical function of the heart; this condition, known as pulseless electrical activity (PEA), is likely due to ischemic stunning. It would be desirable for internal cardioverter-defibrillators (ICD's) to provide PESP in order to augment post-shock resuscitation. One category of ICD's includes systems that are intended for implantation, in their entireties, outside a rib cage of a patient in a subcutaneous space; incorporation of PESP into these sub-Q ICD's presents new challenges.

### SUMMARY

Embodiments of the present invention include sub-Q systems and methods for post-shock delivery of PESP to augment cardiac resuscitation. According to some embodiments, a duration of fast VT or VF in between shocks restoring normal sinus rhythm is logged and used as a criterion for delivery of post-shock PESP therapy. Sub-Q systems of the present invention include a pair of electrodes adapted to deliver electrical stimulation to a heart, for example high voltage shocks and potentiation pulses, and a set of electrodes adapted to sense a response to the potentiation pulses. Some embodiments further include sensing of parameters indicative of cardiac mechanical function.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a schematic of an exemplary sub-Q ICD system implanted in a patient according to some embodiments of the present invention.

FIG. 2 is a flow chart depicting a method of the present invention.

FIG. 3A is a trace illustrating an exemplary situation in which PESP therapy is triggered.

FIG. 3B is a trace illustrating an exemplary situation in which PESP therapy is withheld.

FIG. 4A is a schematic diagram of an amplifier and filter architecture according to one embodiment of the present invention.

FIG. 4B is a block diagram of an exemplary overall system architecture into which the amplifier and filter of FIG. 4A may be integrated.

FIG. 5 is a section view of an optical sensor mounted on a housing of an ICD according to one embodiment of the present invention.

### DESCRIPTION OF VARIOUS EMBODIMENTS

The following detailed description is exemplary in nature and is not intended to limit the scope, applicability, or configuration of the invention in any way. Rather, the following description provides practical illustrations for implementing exemplary embodiments of the present invention.

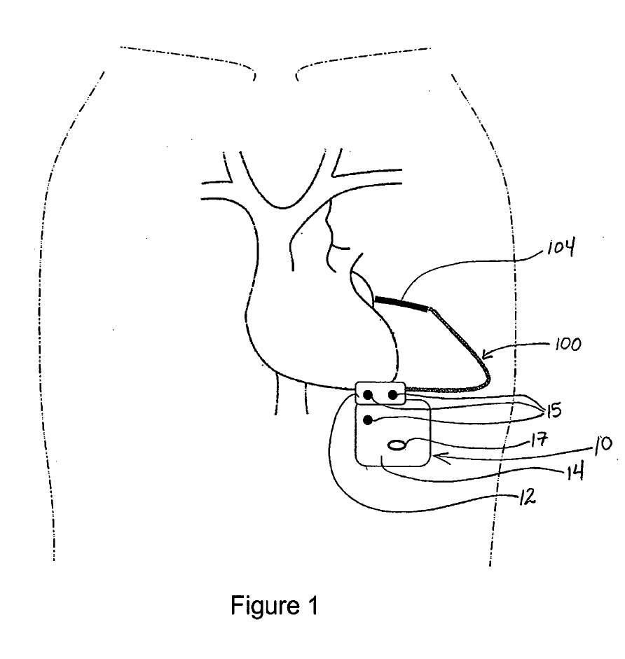

FIG. 1 is a schematic of an exemplary sub-Q ICD system implanted in a patient according to some embodiments of the present invention. Dashed lines in FIG. 1 represent a thorax of the patient. The heart is shown in FIG. 1 for reference; it will be appreciated by those skilled in the art that the system has been implanted exterior to the rib cage, or extra-thoracic, without exposing the heart, according to methods known to those skilled in the art for the implant of sub-Q ICD systems. FIG. 1 illustrates the system including a device 10 to which a medical electrical lead 100 is coupled by a connector module 12 of device 10. Lead 100 is shown including a stimulation electrode 104, which has been implanted at a posterior extra-thoracic site. According to the illustrated embodiment, a housing 14 of device 10 serves as another stimulation electrode to act in concert with electrode 104 for electrical stimulation of the heart; such an electrode is known to those skilled in the art as an ‘active can’. Device 10 is shown implanted anterior to the heart at an extra-thoracic location generally corresponding to the apex of the heart or adjacent a cardiac notch. Lead electrode 104 and device housing 14, disposed opposite one another on either side of the heart, provide an electrical stimulation vector that passes through a sufficient bulk of ventricular muscle mass of the heart to make stimulation, for example, pacing and/or shocking, effective. Those skilled in the art will appreciate that lead electrode 104 has been positioned, via a subcutaneous path traversing around a lateral side of the patient's thorax, just lateral and inferior to a left scapula. It should be noted that sub-Q ICD systems according to the present invention are not limited to the illustrated implant configuration. Furthermore, suitable materials and construction methods for device 10 and lead 100, as well as connection methods for coupling lead 100 to device 10 within connector module 12, are well known to those skilled in the art.

FIG. 1 further illustrates a set of electrodes 15 including two electrodes mounted in a sidewall of device connector module 12 and one electrode mounted in a sidewall of device housing 14; alternately all of electrodes 15 could be mounted in the housing sidewall. Electrodes 15 are shown disposed approximately orthogonal to one another and approximately orthogonal to the stimulation vector extending between housing 14, acting as an electrode, and lead electrode 104. Such an orthogonal arrangement of electrodes 15 provides independent electrocardiogram channels that can enhance sensing capability as described in commonly assigned U.S. Pat. No. 5,331,966, the relevant parts of which are hereby incorporated by reference. Materials and construction methods suitable making and mounting electrodes 15 are known to those skilled in the art and are described, for example, in commonly assigned U.S. Pat. Nos. 4,310,000 and 6,622,046, the relevant parts of which are hereby incorporated by reference.

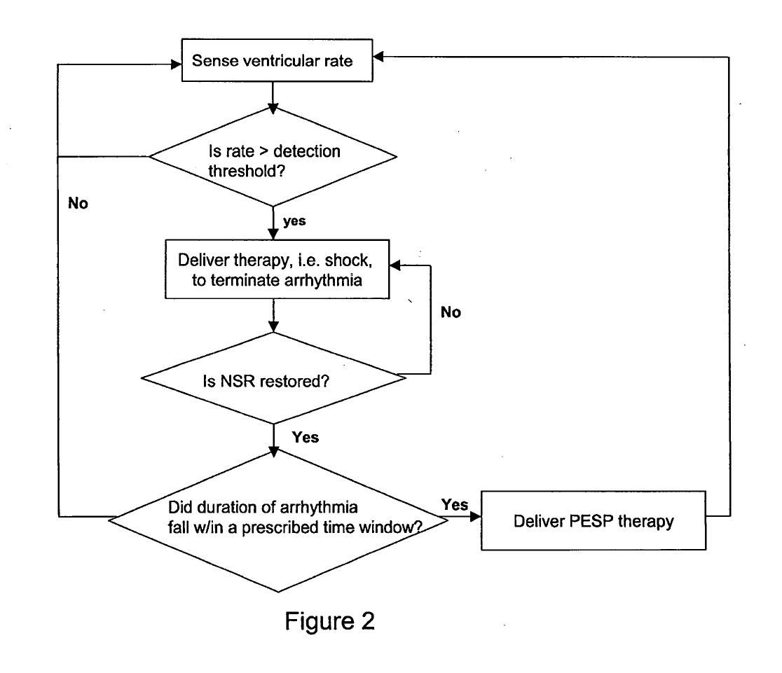

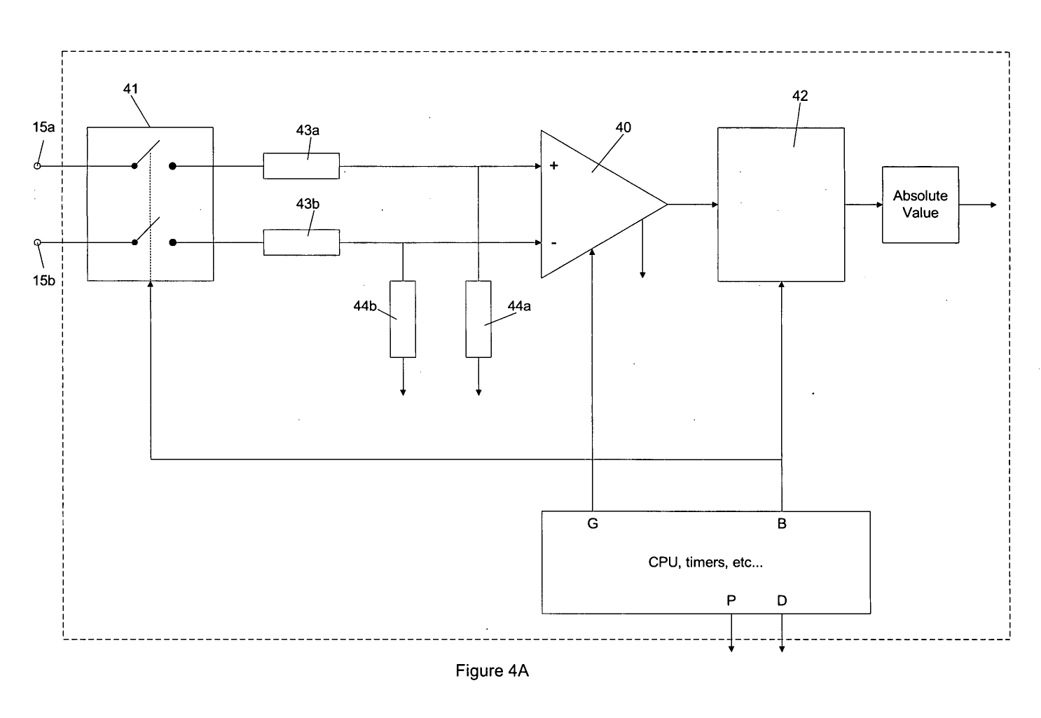

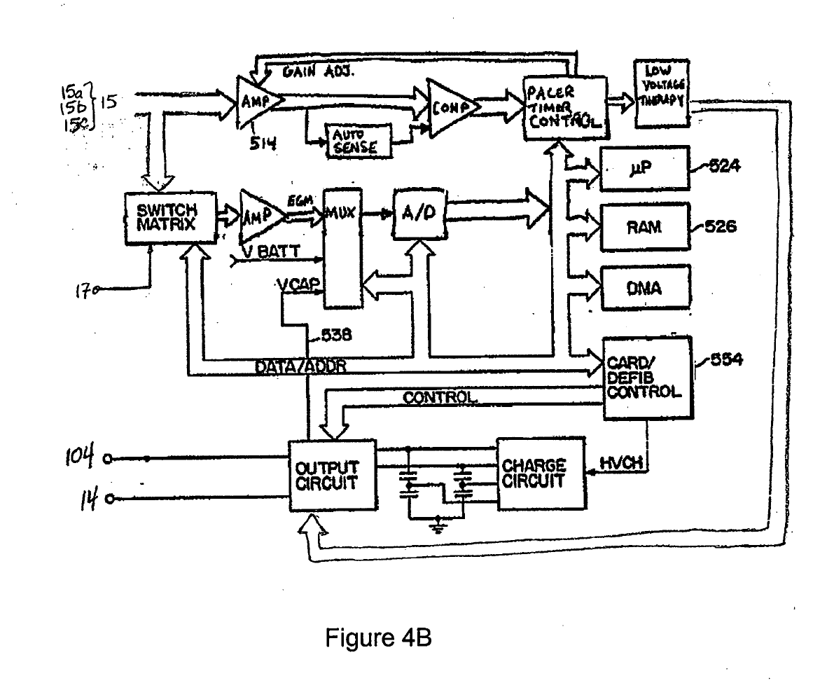

According to embodiments of the present invention, an electrode pair, for example housing electrode 14 and lead electrode 104 illustrated in FIG. 1, provide shocking stimulation for cardiac defibrillation and post-shock cardiac potentiation stimulation, or PESP, to augment post-shock resuscitation when necessary (the pair may further provide pacing stimulation according to some embodiments). Methods of the present invention, for example as illustrated in the flow chart of FIG. 2, determine the necessity of PESP therapy according to a duration of arrhythmic, for example, fast VT or VF, episodes that occur in between shocks restoring NSR; these episodes are characterized, for example, by a rate greater than approximately 250 beats per minute at which a detection threshold is set. According to embodiments of the present invention, for example as illustrated in FIG. 1, ventricular depolarization pulses are sensed by electrodes 15 and logged by a counter that determines the rate of the pulses and a duration of pulses that are categorized as an arrhythmia, for example, fast VT or VF; the counter is included in a microprocessor 524 and/or a RAM element 526, for example, as shown in the exemplary system architecture of FIG. 4B, which are hermetically sealed in housing 14 of device 10.

According to the method described in FIG. 2, if the duration of the arrhythmia falls within a prescribed time window, post-shock PESP therapy is delivered. According to certain embodiments of the present invention, the duration time window is set between approximately 30 seconds and approximately 300 seconds, since it is highly probable that PESP pulses are needed if the duration of the arrhythmia is greater than about 30 seconds, and it is more likely that the pulses will be effective in restoring adequate cardiac mechanical function if the duration of the arrhythmia does not exceed about 300 seconds.

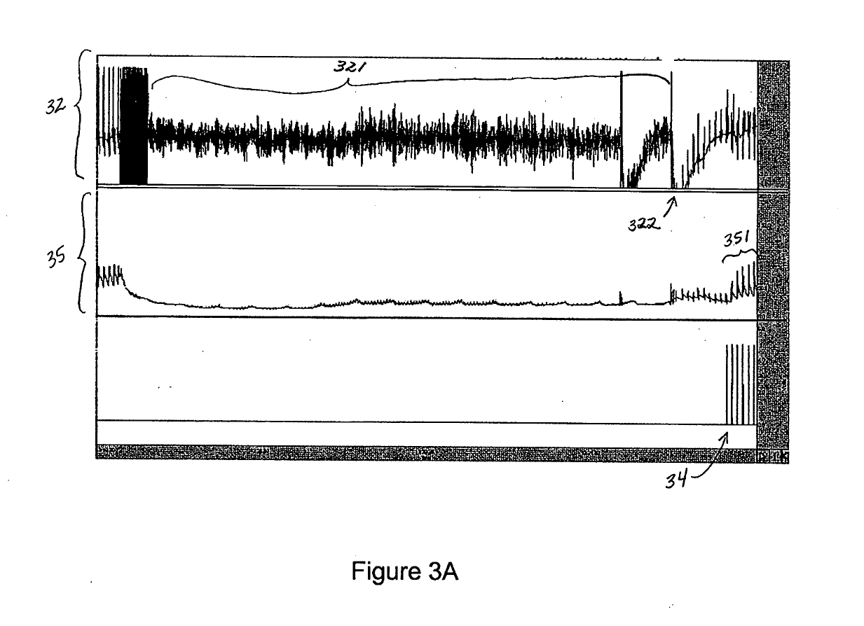

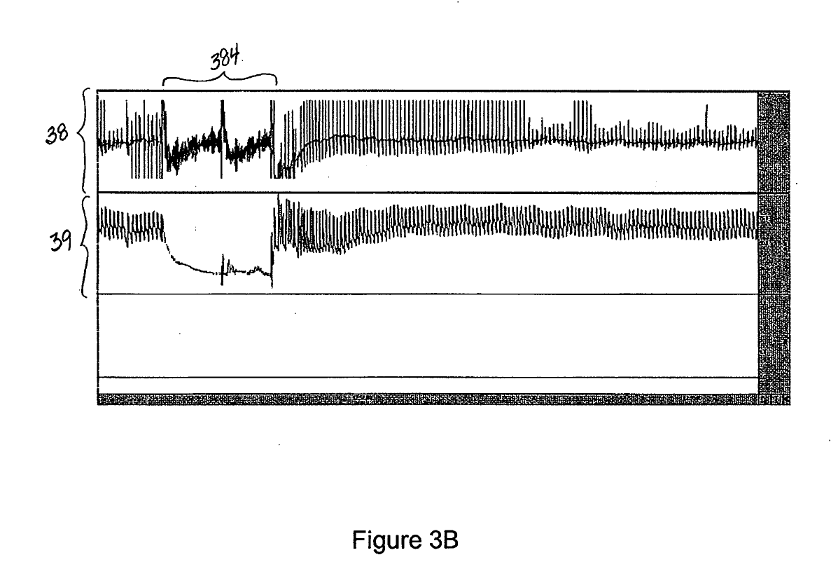

FIG. 3A is a trace illustrating an exemplary situation in which PESP therapy is triggered. FIG. 3A illustrates an electrocardiogram (ECG) trace 32 showing a duration of VF 321 that is terminated at point 322, for example by a high voltage shock delivered between housing 14 and lead electrode 104 (FIG. 1). According to the illustrated example, the duration of VF 321 is within a prescribed time window, for example between approximately 30 seconds and approximately 300 seconds, triggering the delivery of PESP pulses 34, as shown in the lower most trace. A trace of pulse pressure 35 indicates poor post-shock recovery of cardiac mechanical function, thus confirming the need for post-shock PESP therapy; and the efficacy of the post-shock PESP pulses is illustrated by an increase in pulse pressure 351. According to some embodiments of the present invention, a duration of PESP therapy may be set according to a predetermined or prescribed time limit, for example 30 seconds, or may be responsive to sensing of adequate cardiac mechanical function, for example by sensing of the pulse pressure as recorded in trace 35 or by sensing other signals indicative of cardiac mechanical function, which will be described below. Additionally, PESP therapy may be terminated upon detection of VT or VF.

FIG. 3B is a trace illustrating an exemplary situation in which PESP therapy is withheld. FIG. 3B illustrates an ECG trace 38 wherein a duration of VF 384 is outside a prescribed time window, for example less than 30 seconds, so that PESP therapy is not triggered. Such a short duration of VF is unlikely to result in ischemic stunning of the heart that can cause PEA, and a pulse pressure trace 39 confirms this, showing good post-shock recovery of mechanical function without the need for PESP therapy.

According to embodiments of the present invention, after determining the need for PESP therapy, sensing by electrodes 15 (FIG. 1) is used to set an amplitude of PESP pulses and a timing of the pulse delivery. Electrodes 15 sense ventricular depolarization to trigger the pulses and then, approximately within the first 100 to 200 milliseconds post-pulse, sense for a response evoked by the pulse, which would indicate that the therapy pulse captured the heart. Upon sensing an evoked response, the system can establish an effective timing and amplitude for PESP pulses. According to some embodiments, the system selects a pair of electrodes from among electrodes 15, for such sensing; the pair may be one which has provided adequate sensed VT signal amplitudes. According to alternate embodiments, all of electrodes 15 may used to provide a composite signal, for example a square root sum of squares.

According to some embodiments of the present invention, to enhance sensing of evoked responses, electrodes 15 each include an enlarged microscopic surface area that reduces polarization and thus increases sensitivity of each electrode to response signals evoked by PESP pulses. A coating, formed from example by sintering or sputtering, may provide the enlarged surface; suitable coating materials include, but are not limited to, platinum, platinum black, titanium nitride and ruthenium oxide. Those skilled in the art will appreciate that each electrode of electrodes 15 is electrically isolated from the other and from housing 14 and from lead electrode 104 and that each of electrodes 15 is electrically coupled, via a feedthrough (not shown) to an input terminal of a sense amplifier (not shown) that is hermetically sealed within housing 14. A system architecture including a sense amplifier having relatively fast recovery properties to further facilitate sensing of responses evoked by PESP pulses, according to one embodiment of the present invention, is described in conjunction with FIGS. 4A-B.

FIG. 4A is a schematic diagram of a fast recovery amplifier and filter architecture according to one embodiment of the present invention; and FIG. 4B is a block diagram of an exemplary overall system architecture into which the amp and filter of FIG. 4A may be integrated, for example as one amplifier channel of AMP 514 shown in FIG. 4B. It should be recognized that all of electrodes 15 would be coupled into the system, as shown in FIG. 4B, according to the manner depicted for electrodes 15a and 15b in FIG. 4A, but, to keep FIG. 4A relatively simple, only the two electrodes 15a, 15b are shown.

FIG. 4A illustrates electrodes 15a, 15b coupled to an instrumentation amplifier 40 through a blanking isolation circuit 41, including FET switches. According to the illustrated embodiment, amplifier 40 is coupled to a bandpass filter 42 and both amp 40 and filter 42 receive gain G and blanking B information from timers that control pacing pulses P, including PESP pulses, and defibrillation pulses D, for example, in blocks 515 and 554, respectively, of FIG. 4B. According to preferred embodiments, amplifier 40 has programmable gain, excellent common mode rejection properties and fast transient recovery. Bandpass filter 42 may include a reset/hold function for rapid recovery after a step change in input following a blanking interval.

FIG. 4A further illustrates coupling elements 43a and 43b connected in series between respective electrodes 15a, 15b and amplifier 40, downstream of blanking isolation circuit 41; each element 43a, 43b includes a capacitive coupling element and a series resistance, and may further include a current clamp. The current clamp may be a current limiting semiconductor, to assure that external high voltage pulses, i.e. defibrillation shocks, do not result in excessive current being conducted back through electrodes 15a, 15b that could damage tissue or produce relatively large polarization voltages. Shunt elements 44a and 44b are shown coupled to respective electrodes 15a, 15b; elements 44a, 44b consist of voltage clamps and high impedance pathways to an analog ground to define a reference ground.

Referring back to FIG. 1, device 10 further includes a sensor 17 mounted on housing 14; according to some embodiments of the present invention sensor 17 provides feedback indicative of cardiac mechanical function. Thus, the feedback indicative of cardiac mechanical function may be used in conjunction with cardiac electrical signals, sensed by electrodes 15, to better determine if post-shock PESP therapy is required and/or when to terminate PESP therapy.

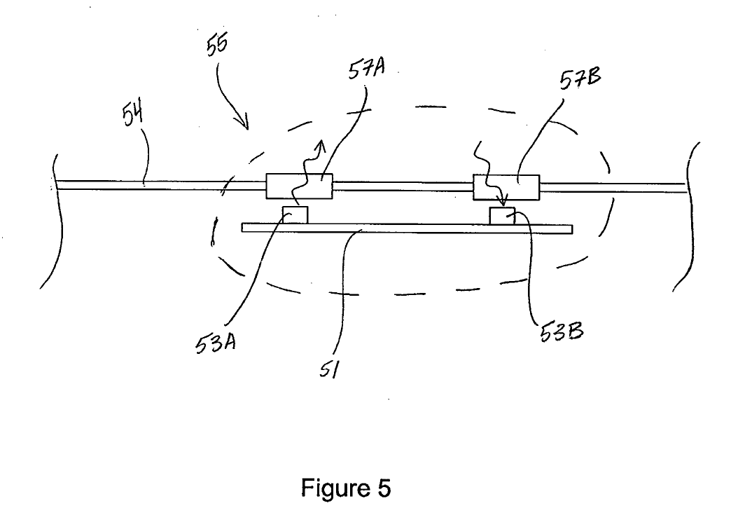

A type of sensor particularly suited to a wholly sub-Q system, for example as illustrated in FIG. 1, is that which, being implanted at a location remote from the heart, can pick up signals indicative of cardiac mechanical function. Examples of this type of sensor include, but are not limited to, an optical sensor for sensing tissue oxygenation indicative of perfusion or pulse pressure, an acoustic sensor for sensing heart sounds, and an accelerometer for sensing motion of the chest wall indicative of cardiac pumping action. An exemplary device including sensor 17 in the form of an optical sensor 55 is described in conjunction with FIG. 5.

FIG. 5 is a section view of optical sensor 55 (surrounded by dashed lines) mounted on a portion of a housing sidewall 54, for example formed of titanium, of an ICD according to one embodiment of the present invention. FIG. 5 illustrates sensor 55 including a pair of optical windows 57A and 57B, each mounted in sidewall 54; each window 57A, 57B may be formed of sapphire and coupled to sidewall 54 by a hermetic braised ferrule seal. FIG. 5 further illustrates sensor 55 including a photon source 53A, for example an LED or laser diode, disposed within housing sidewall 54, beneath window 57A, and a photodetector 53B, for example a diode or CCD, also disposed within sidewall 54, beneath window 57B; photon source 53A and photodetector 53B are each mounted on an optics circuit board 51 that drives photon source 53A and conditions signals from photodetector 53B.

FIG. 5 shows photons emitted from source 53A traveling out window 57A to adjacent tissue (not shown) where the photons reflect, scatter, and are absorbed according to a wavelength of the photons and a state of perfusion within the adjacent tissue. A portion of the reflected photons is shown returning through window 57B to photodetector 53B; opto-electronic circuits of circuit board 51, which are coupled to photodetector 53B, provide light intensity information. According to some embodiments of the present invention, photon source 53A emits two or more wavelengths suitable for determining when insufficient levels of oxygen are in the adjacent tissue, indicative of hypo-perfusion, and when adequate levels of oxygen are in the adjacent tissue, indicative of adequate perfusion resulting from pulsatile blood flow. Alternate embodiments of sensor 17 include multiple photon sources that each emit multiple wavelengths, and corresponding detectors, to directly determine a magnitude of pulsatile blood flow in adjacent tissue. Such information, concerning oxygenation and pulsatile flow, can be linked to the condition of cardiac mechanical function, for example indicating when the heart is, or is not, pumping at a high enough pressure to provide adequate blood flow. According to certain embodiments of the present invention, this perfusion information is processed and used in conjunction with the previously described cardiac electrical signals to trigger or to withhold PESP therapy and, once PESP therapy has been initiated, to determine when the therapy can be terminated. With reference to FIG. 4B, signals from electrodes 15 and sensor 17 are shown input into the system for processing.

In the foregoing detailed description, the invention has been described with reference to specific embodiments. However, it may be appreciated that various modifications and changes can be made without departing from the scope of the invention as set forth in the appended claims.

1. A computer-readable medium programmed with instructions for performing a method of augmenting cardiac resuscitation in a sub-Q ICD system, the medium comprising instructions for causing a programmable processor to:
log a duration of fast VT or VF between shocks delivered by the system that restore normal sinus rhythm;
deliver post-shock post-extra-systolic potentiation pulses dependent upon the restoration of normal sinus rhythm and the logged duration being within a prescribed time window;
sense for an electrical response to the potentiation pulses; and
adjust a timing of the potentiation pulses according to the sensing.

2. The medium of claim 1, further including instructions to:
sense for a parameter indicative of cardiac mechanical function; and
terminate the potentiation pulses based on detection of the sensed parameter.

3. The medium of claim 2, wherein the sensing for the parameter indicative of cardiac mechanical function is accomplished by a sensor implanted at a location remote from the heart.

4. The medium of claim 2, wherein the parameter includes at least one of tissue oxygenation indicative of an arterial pulse, heart sounds, and chest wall motion indicative of cardiac pumping action.

5. The medium of claim 1, wherein the prescribed time window extends between approximately 30 seconds and approximately 300 seconds.

6. The medium of claim 1, further including instructions to terminate the potentiation pulses after a prescribed time.

7. The medium of claim 6, wherein the prescribed time is approximately 30 seconds.

8. The medium of claim 1, further including instructions to terminate the potentiation pulses upon detection of VT or VF.

9. The medium of claim 1, further including instructions to sense for a parameter indicative of cardiac mechanical function, and wherein delivering the potentiation pulses is further dependent upon sensing the parameter.

10. The medium of claim 1, further including instructions to select a pair of sensing electrodes from a set of orthogonal electrodes to sense the electrical response to the potentiation pulses.

11. The medium of claim 10, wherein the selected pair of sensing electrodes corresponds to a pair, from among the set of orthogonal electrodes, that provides a maximum signal amplitude of sensed VF.

12. The medium of claim 1, further including instructions to adjust an amplitude of the potentiation pulses according to the sensing.

13. An ICD system adapted for subcutaneous implantation, the system comprising:
a pair of electrodes adapted to deliver electrical stimulation to a heart;
a set of low polarization electrodes for sensing a response to electrical potentiation pulses delivered from the first pair of electrodes; and
a counter for logging a duration of fast VT and VF between high voltage shocks that restore normal sinus rhythm to the heart, the shocks delivered by the first pair of electrodes.

14. The system of claim 15, further comprising an optical sensor adapted to sense tissue oxygenation indicative of an arterial pulse.

15. The system of claim 14, further comprising a housing containing some electrical components of the system, and wherein the optical sensor is mounted in a sidewall of the housing.

16. The system of claim 13, wherein the set of electrodes includes three electrodes disposed orthogonal to one another.

17. The system of claim 16, further comprising a housing containing some electrical components of the system, and wherein at least one of the three electrodes is mounted in a sidewall of the housing.

18. The system of claim 13, further comprising:
a housing containing some electrical components of the system, the housing serving as a first electrode of the pair of electrodes; and
an electrical lead including an electrode serving as a second electrode of the pair of electrodes.

19. An implantable medical device system, comprising:
a pair of electrodes adapted to deliver electrical stimulation to a heart;
a set of electrodes for sensing a response to electrical potentiation pulses delivered from the first pair of electrodes, the set of low polarization electrodes being orthogonal to each other; and
a counter for logging a duration of fast VT and VF between high voltage shocks that restore normal sinus rhythm to the heart, the shocks being delivered by the first pair of electrodes.

20. The ICD system of claim 19, wherein the set of electrodes includes a pair of low polarization electrodes.
